(19) United States
(12) Patent Application Publication
(10) Pub. No.: US 2004/0233387 A1
(43) Pub. Date: Nov. 25, 2004

(54) Systems and methods for analysis of corneal topography with convexity map

(76) Inventors: David Huang, Cleveland Heights, OH (US); Raj Shekhar, Sagamore Hills, OH (US); Maolong Tang, Columbus, OH (US)

Correspondence Address: Richard S Wesorick, Tarolli Sundheim Covell & Tummino, Suite 1111, 526 Superior Avenue, Cleveland, OH, 44114, US

(21) Appl. No.: 10/493,177

(22) PCT Filed: Oct. 18, 2002
(86) PCT No.: PCT/US02/33294

(30) Foreign Application Priority Data
Oct. 18, 2001 (US) 60330391

(51) Int. Cl. A61B 3/107; A61B 18/18

(52) U.S. Cl. 351/212; 606/5

(57) ABSTRACT

Systems and methods provide convexity map data associated with captured surface image data of a cornea The convexity map data is determined by transforming an elevation map data set into a convexity map data set. Convexity is computed as the negative of the Laplacian of the local elevation. One or more statistical parameters can be associated with the convexity map data set and employed to derive indices. The indices can be utilized to diagnosis a corneal condition.

### CROSS-REFERENCE TO RELATED APPLICATIONS

The present application claims the benefit of U.S. Provisional Patent Application Ser. No. 60/330,391, filed Oct. 18, 2001, entitled METHOD FOR ANALYSIS OF CORNEAL TOPOGRAPHY WITH CONVEXITY MAP, and which is incorporated herein by reference.

### TECHNICAL FIELD

The present invention relates to shape measurements of the eye, and more particularly to systems and methods for analysis of corneal topography with a convexity map.

### BACKGROUND OF THE INVENTION

Computer-assisted videokeratography (e.g., corneal topography) has become an extensively used clinical tool to measure the shape of the anterior surface of a human cornea. Typical corneal topographers are based on a placido-ring technique. A placido-ring technique includes placing a series of concentric rings (ie., the Placido rings) mounted on a placido disk-type nose cone in front of the cornea to be measured. A light source illuminates the placido disc. The rays reflected off the cornea are captured by a camera (e.g., charge-coupled device (CCD) camera), and the reflection pattern of the rings are analyzed by a processor to reconstruct the corneal shape automatically. The reflection of the rings is obtained from the corneal surface, for example, by viewing, photographing or videographing the corneal image through a hole in the center of the disc. The shape of the cornea can be mapped to a graphical display and/or the data analyzed for diagnosis of one or more corneal conditions. Although most methods for assessing corneal topography are based on the principle of reflection, other techniques employ projection. Additionally, wave-front analysis can now provide information about the refractive power of the eye as a whole, rather than just the effect of the anterior corneal surface.

Placido-ring based corneal topography systems are most commonly used to measure the corneal curvature. The resulting data is currently processed to yield maps of axial (e.g., sagittal) power and tangential (e.g., instantaneous curvature) power. Both types of maps are based on a spherical model of the cornea with a pre-defined central axis. However, the axial power map and the tangential power map have limitations in the diagnosis of certain corneal defects. For example, certain characteristics (e.g., corneal conditions, measurement errors) associated with axial power maps and tangential power maps reduce the ability to diagnose certain corneal defects, such as keratoconus. Keratoconus is a non-inflammatory corneal disease where areas of the cornea thins and bulges (become “ectatic”) over time.

The axial power map and the tangential power map are measures of radial slope (axial) or curvature (tangential) and ignore elevation variation in the azimuthal direction. Therefore, they depend on the proper alignment of the eye. Misalignment changes the assumed central axis and can make a normal eye appear abnormal. Also, regular astigmatism causes changes on these maps which confound the diagnosis of keratoconus. Furthermore, due to the employment of a spherical model to map the cornea, which is not a perfect sphere, the appearance of the cone is less pronounced off center. This effect is particularly severe on axial maps. Laser ablation centration is currently manually traced on axial or tangential map displays. Laser ablation presence is determined by visual inspection of axial or tangential maps, which can be effected by certain characteristics associated with the axial or tangential maps.

Refractive surgeons, corneal specialists and ophthalmologists in general will use corneal topographers to improve the safety of Laser in-situ Keratomileusis (LASIK), the quality of vision after LASIK and to diagnose corneal conditions. LASIK is the most popular refractive surgery procedure with annual case volume of close to 1 million in the United States. Prior to initial LASIK, it is important to detect patients with keratoconus and forme fruste keratoconus (i.e., subclinical early stage of keratoconus). These patients should not have LASIK, which could exacerbate keratoconus and cause visual symptoms of blurriness and distortion. Certain corneal conditions (e.g., laser ablation, astigmatism) effect the axial and/or tangential map displays causing difficulty when trying to determine corneal characteristics, such as if a patient is a candidate for LASIK.

### SUMMARY OF THE INVENTION

The following presents a simplified summary of the invention in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

Systems and methods are employed to provide convexity map data associated with captured surface image data of a cornea. The convexity map data is determined by transforming an elevation map data set into a convexity map data set. The data points in the elevation map data set are transformed from a global coordinate data set to a local coordinate data set with the Laplacian of the local data points determined to provide a convexity value associated with the local elevation data points. Convexity is computed as the negative of the Laplacian of the local elevation. Convexity is a characteristic of local elevation variation and is equal to the inverse of the radius of the sphere that best fits the local surface. One or more statistical parameters can be associated with the convexity map data set and employed to derive indices (e.g., maximum, minimum, median, inferior hemi-averages, superior hemi-averages, max-median difference, max-min difference, inferior-superior difference). The indices can be utilized to diagnose a corneal condition (e.g., normal cornea, cornea with keratoconus, cornea with previous keratorefractive surgery).

In one aspect of the invention, a corneal topography system is provided that includes an optical assembly that captures image of a cornea that is converted to image data. A diagnostic system converts the image data to elevation map data. A convexity module then converts the elevation map data to convexity map data. A module can be an algorithm, function, or computation that is provided by software and/or hardware. The convexity module can transform the elevation map data points from global coordinates to local coordinates employing a transform matrix. The local elevation map is then convolved with a matrix kernel to perform the Laplacian operation. The results of the Laplacian operation can be employed to produce a map of convexity values. The convexity values can be employed in the diagnosis of a corneal condition (e.g., keratoconus or previous keratorefractive surgery) in addition to providing other information (e.g., center of laser ablation) associated with the cornea.

The convexity module and other modules of the diagnostic system can be software modules residing as computer executable instructions residing on a computer readable medium (e.g., hard drive, floppy disk, CD-Rom). The convexity module can be integrated with other diagnostic software modules as part of a complete cornea diagnostics and display system. Alternatively, the convexity module can be a stand-alone diagnostics and display program.

The following description and the annexed drawings set forth certain illustrative aspects of the invention. These aspects are indicative, however, of but a few of the various ways in which the principles of the invention may be employed. Other advantages and novel features of the invention will become apparent from the following detailed description of the invention when considered in conjunction with the drawings.

### BRIEF DESCRIPTION OF THE DRAWINGS

FIG. 1 illustrates a block diagram of a system for mapping corneal image data in accordance with an aspect of the present invention.

FIG. 2 illustrates a block diagram of a corneal topography system in accordance with an aspect of the present invention.

FIG. 3 illustrates a block diagram of a mapping system in accordance with an aspect of the present invention.

FIG. 4 illustrates a map display set of a map simulation of a central cone (5D) on a regular cornea without astigmatism.

FIG. 5 illustrates a map display set of a simulation of an inferior-temporal cone (5D) on a regular cornea with the cone moved to the mid-peripheral region.

FIG. 6 illustrates a map display set of a simulation of an inferior-temporal cone (5D) on 4D with-the-rule astigmatism.

FIG. 7 illustrates a map display set of an actual topography from a keratoconic eye.

FIG. 8 illustrates a block diagram of a system for determining the presence of keratoconus in a cornea in accordance with an aspect of the present invention.

FIG. 9 illustrates a block diagram of a system for locating a center of laser ablation in accordance with an aspect of the present invention.

FIG. 10 illustrates a block diagram of a system for detecting the presence of previous keratorefractive surgery of a cornea in accordance with an aspect of the present invention.

FIG. 11 illustrates a graphical display of a convexity map of a cornea with keratoconus.

FIG. 12 illustrates a graphical display of a convexity map of a simulated cylinder without keratoconus.

FIG. 13 illustrates a graphical display of a convexity map of a planned ablation profile.

FIG. 14 illustrates a graphical display of a convexity map of an elevation change map.

FIG. 15 illustrates a graphical display of a convexity map of a normal Pre-LASIK patient.

FIG. 16 illustrates a graphical display of a convexity map of a normal Post-LASIK patient.

FIG. 17 illustrates a flow diagram of a methodology for diagnosis of a condition associated with a cornea in accordance with an aspect of the present invention.

FIG. 18 illustrates a block diagram of a suitable computing environment in which various aspects of the present invention can be implemented.

### DETAILED DESCRIPTION OF THE INVENTION

The present invention relates to corneal topographic measurements, and the diagnosis of corneal conditions (e.g., healthy, diseases, shape abnormalities, distortions) employing the corneal topographic measurements. In particular, the present invention relates to systems and methods for proving a convexity map data set which provides convexity information or values about different location points on the surface of the cornea. The convexity information can be determined by applying the Laplacian operation on an elevation map data set. Convexity is computed as the negative of the Laplacian of the local elevation. Convexity is a characteristic of local elevation variation and is equal to the inverse of the radius of the sphere that best fits the local surface.

In one aspect of the present invention, each of the convexity data points are calculated from a group of points covered in the area of a convolution kernel. The present example illustrates employing a kernel covering a square area of nine points. However, it is to be appreciated that other methodologies can be employed to perform the Laplacian operation on an elevation map data set to provide the desired convexity information.

One or more statistical parameters can be associated with the convexity map data set. The one or more statistical parameters can be employed to derive indices. The indices can be utilized to diagnosis a corneal condition (e.g., healthy, keratoconus, previous keratorefractive surgery, shape abnormalities or distortions). Additionally, the convexity map data set can be graphically displayed along with an index indicating different levels of convexity. The convexity map display can be provided with other corneal topographic measurement displays (e.g., axial map, tangential map, elevation map) in a corneal topographic system.

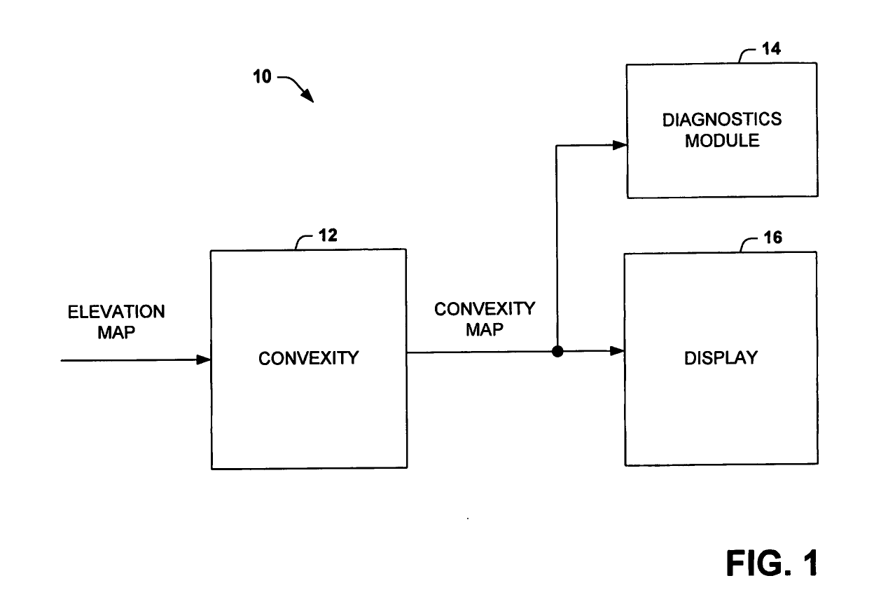

FIG. 1 illustrates a system 10 for mapping corneal image data in accordance with an aspect of the present invention. The system 10 includes a convexity module or function 12 that receives elevation map data associated with a corneal image and provides a convexity map. The elevation map data can be derived from axial radius data associated with a corneal image provided by an image capturing system. Alternatively, the elevation data can be provided directly from the imaging capturing system. Convexity is the negative Laplacian of local elevation, which can be represented as:

$$- {∇}^{2} h = - ( \frac{{\partial }^{2} h}{\partial {x}^{2}} + \frac{{\partial }^{2} h}{\partial {y}^{2}} ) \qquad EQ . 1$$

where ∇² is the Laplacian operation in two dimension x and y, and where h represents the corneal surface elevation along the z axis and (x, y, z) is the local rectangular coordinate system with origin at a point on the corneal surface with z being the normal to the corneal surface. The convexity module 12 can be estimated from the difference between the surface height at a center point and the surface height over eight rectangular neighboring points.

At each point on the elevation map, the elevation data on that point and neighboring points is transformed from the global to the local coordinate system and the Laplacian operation performed on the local coordinate. The global coordinate system has its central (z) axis along the eye's line-of-sight and origin at the intersection of the central axis with the anterior corneal surface. The local coordinate system defines the z-axis as being normal to the surface at the data point, and placing the origin at the local data point. As an analogy, a global coordinate system for Earth would have the z-axis as the axis of rotation and an origin at the north pole. The local coordinate system on Earth would have a z-axis defined as normal to the local sea surface and measured height from local sea level.

The coordinate transformation can be determined by the following analysis. As is known, any rotation of space fixing the origin can be decomposed as a rotation by φ about the x-axis, followed by a rotation by θ about the y-axis, followed by rotation by ψ about the z-axis. The numbers φ, θ and ψ are called the Euler angles of the composite rotation, which acts as follows:

(x,y,z)→M(x,y,z), EQ. 2

where M is the matrix given by EQ. 3 as follows:

[cos φ cos θ cos ψ−sin φ sin ψ−sin φ cos θ cos ψ−cos φ sin ψ sin θ cos ψ cos φ cos θ sin ψ+sin φ cos ψ−sin φ cos θ sin ψ+cos φ cos ψ sin θ sin ψ−cos φ sin θ sin φ sin θ cos θ] EQ. 3

If z transforms to (Nₓ, N_{y}, N_{z}), and Nₓ²+N_{y}²+N_{z}²=1, which denote the local surface normal, then:

$$φ = - arccos ( \frac{{N}_{x}}{\sqrt{{N}_{x}^{2} + {N}_{y}^{2}}} ) ,$$

θ=−arccos N_{z}, ψ=arbitrary (0 is the default) EQ. 4

$$M = [ \begin{matrix}\frac{{N}_{x} {N}_{z}}{\sqrt{{N}_{x}^{2} + {N}_{y}^{2}}} & \frac{{N}_{y} {N}_{z}}{\sqrt{{N}_{x}^{2} + {N}_{y}^{2}}} & - \sqrt{{N}_{x}^{2} + {N}_{y}^{2}} \\ - \frac{{N}_{y}}{\sqrt{{N}_{x}^{2} + {N}_{y}^{2}}} & \frac{{N}_{x}}{\sqrt{{N}_{x}^{2} + {N}_{y}^{2}}} & 0 \\ {N}_{x} & {N}_{y} & {N}_{z}\end{matrix} ] \qquad EQ . 5$$

Using the transform matrix of M, the direction of elevation (z axis) can be rotated to the surface normal of each data point. Therefore, the Laplacian computed in the transformed coordinates denotes the local convexity.

The local data for each data point is then used to compute convexity by performing the Laplacian operation. The Laplacian operation is carried out on the data matrix by convolution with a kernel matrix. Given the local coordinates of a given point on the corneal surface, a 3*3 convexity kernel can be employed to compute the convexity. EQ. 6 illustrates one example of a kernel that can be employed in accordance with an aspect of the present invention.

$${f}_{k} = \frac{1}{( 6 * {d}^{2} )} [ \begin{matrix}0.5 & 2 & 0.5 \\ 2 & - 10 & 2 \\ 0.5 & 2 & 0.5\end{matrix} ] \qquad EQ . 6$$

where d is the distance between two adjacent points on a digital mesh or matrix that represent the map data set (i.e., image resolution).

The convexity map can be employed in the diagnosis of keratoconus and forme fruste keratoconus. Additionally, the centration of laser corneal ablation can be determined based on the convexity map. Furthermore, the convexity map can be employed to detect the presence of laser corneal ablation. The convexity map can be provided to a diagnostics module 14 that can employ one or more statistical parameters of the convexity map to diagnosis one or more corneal conditions. Additionally, the convexity map can be provided to a display 16 for providing a graphical display map of the convexity map.

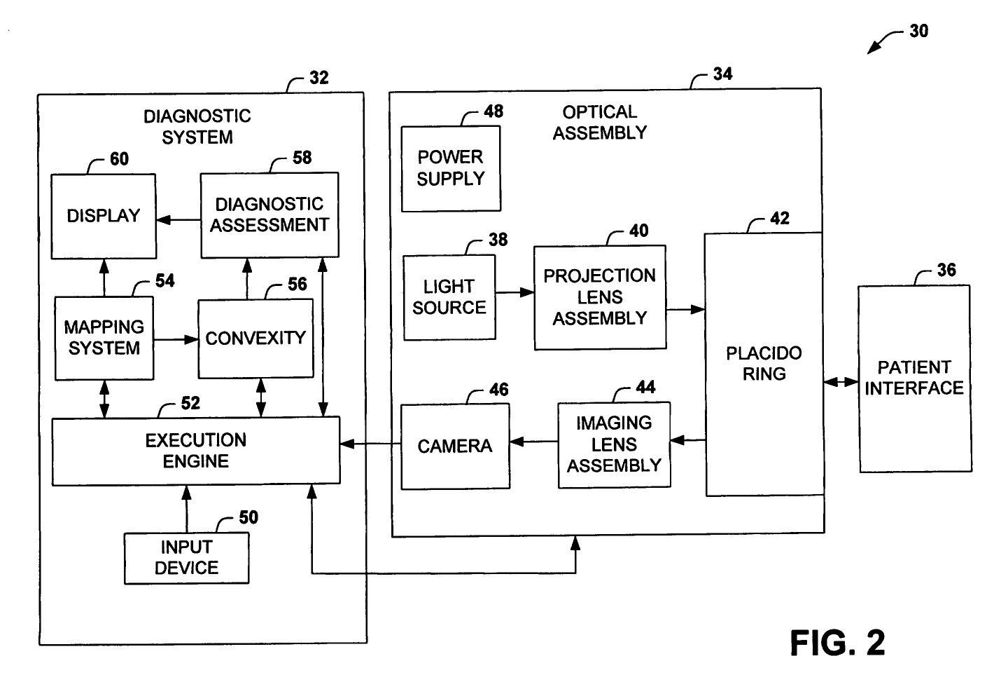

FIG. 2 illustrates a corneal topography system 30 in accordance with an aspect of the present invention. The corneal topography system 30 includes a patient interface 36, an optical assembly 34 and a diagnostic system 32. The diagnostic system 32 can be a personal computer or a stand-alone control device. The patient interface 36, for example, can include a chin rest that can be adjusted based on the patient size with respect to chin to eye dimension. The adjustment can be a screw or slide operating device. The optical assembly 34 can be movable via slides or rollers that can move the optical assembly 34 in two or more dimensions, so that either eye can be aligned on an optical axis and the cornea brought into focus of the optical assembly 34. In the example, of FIG. 2, the diagnostic system 30 controls the operation of the optical assembly 34 and can also control the operation of the slide or rollers. Alternatively, the slide or roller can be operated via manual controls. The patient is placed at the patient interface in front of a placido ring 42 of the optical assembly 34. Adjustments are made such that the patient's cornea is within a viewing space of the optical assembly 42. The patient is requested to focus his eye on a fixation target to assure the coincidence of the optical axes of the instrument with the eye. An image of the cornea can be provided in a display 60 and the adjustments refined so that the entire cornea is viewable within the viewing space.

After the positioning and focusing of the cornea, the optical assembly 34 is activated via an input device 50. The input device 50 can be a foot operated switch, a keyboard or a computer mouse. The activation of the optical assembly 34 captures an image of the cornea. During activation of the optical assembly 34, the placido ring 42 is illuminated from behind by a light source 38, such as a fluorescent tube or the like. The light is projected by the light source 38 through a projection lens assembly 40. The placido ring 42 is reflected from the surface of the cornea, and the reflection is imaged on an electronic camera. The reflection is focused onto the camera 46 by an imaging lens assembly 44. The electronic camera 46 can include an array of photodetectors, such as a CCD with associated electronics and optics. A power supply 48 provides power to the light source 38, the camera 46 and the rollers or slides (not shown).

The diagnostic system 32 includes an execution engine 52 that processes the corneal reflection image. The mapping system 54 can then convert the axial radial power data map to an elevation data map set. Alternatively, the elevation map data set can be provided directly by the mapping system 54. A tangential power map data set can also be provided by converting the radial power map data. The axial power map data set, the tangential power map data set and the elevation map data set can be graphically displayed as topographic maps on a display 60. A convexity module 56 can retrieve elevation data and determine a convexity map data set. The convexity map data set is provided to a diagnostic assessment module 58. The diagnostic assessment module 58 can perform statistical analysis on the convexity map data set to determine one or more corneal conditions. The convexity map data set can be graphically displayed as a topographic map on the display 60. Additionally, the one or more diagnostic conditions can be displayed on the display 60, or retrievable via a diagnostic application program executable by the execution engine 52.

It is to be appreciated that the execution engine 52 can be a diagnostic application program residing in memory in combination with a processor that can execute instructions of the diagnostic application program. Although the example of FIG. 2 illustrates a placido ring cornea topography system, the present invention can be employed on other cornea topography system (e.g., interferometry systems, fluorescence systems).

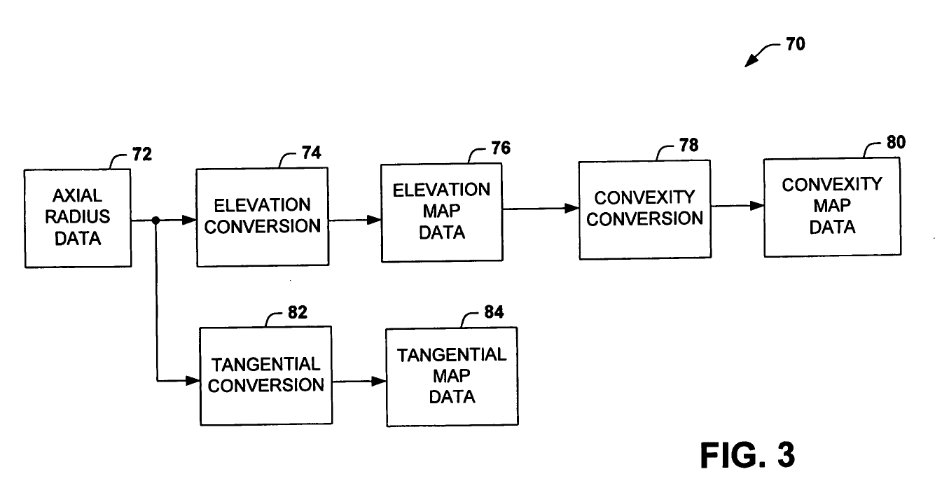

FIG. 3 illustrates an exemplary mapping system 70 in accordance with an aspect of the present invention. The mapping system 70 generates axial radius data 72 via measurements sampled from a CCD camera. The axial radius data set 72 includes axial topographic measurements with respect to a central axis through the center of the cornea and radial distance measurements from the central axis. An elevation conversion module 74 converts the axial radius data set 72 to an elevation map data set 76. Some placido-ring based corneal topography system output elevation maps derived by numerical integration using, for example, the arc-step reconstruction algorithm. However, many system only outputs axial distance maps. The axial distance output can be converted to elevation by computing the following equation:

$$h = {\int }_{0}^{x} tan ( θ ) ⅆ x \qquad EQ . 7$$

where x is distance from a VK (videokeratoscopy) axis and tan(θ) is the surface slope of each data point. Therefore, the elevation conversion module 74 can determine the elevation map data 76 using a numerical integral technique (e.g., segmented Simpson formula). The above integration is done along each meridian. Thus, the anterior surface of the cornea can be reconstructed by fitting all the meridians.

The axial radius data 72 can also be employed to determine a tangential map data 84 using a tangential conversion function 82. The tangential conversion function 82 simply applies the derivative to the axial radius data 72 to provide the tangential map data 84. The elevation map data 76 can be converted to convexity map data 80 by executing a convexity conversion 78 on data points in the elevation map data set 76. The convexity conversion 78 first transforms the data points from global coordinates to local coordinates. The local coordinates are then convolved to provide the convexity map data set 80.

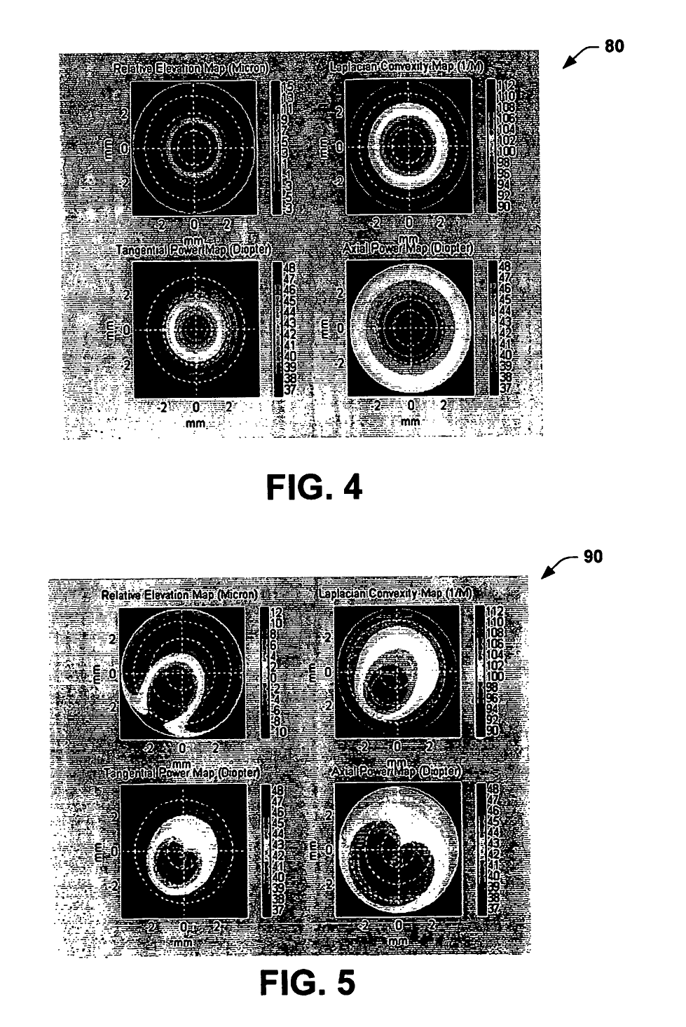

FIGS. 4-7 illustrate different map display set representations of corneal topography for different corneal conditions. Each of the map display sets in FIGS. 4-7l includes a relative elevation map, a convexity map, a tangential power map and an axial power map. FIG. 4 illustrates a map display set 80 of a map simulation of a central cone (5D) on a regular corneal without astigmatism. For the central cone, all maps in the map display set 80 illustrate similar central ectasia FIG. 5 illustrates a map display set 90 of a simulation of an inferior-temporal cone (5D) on a regular cornea with the cone moved to the mid-peripheral region.

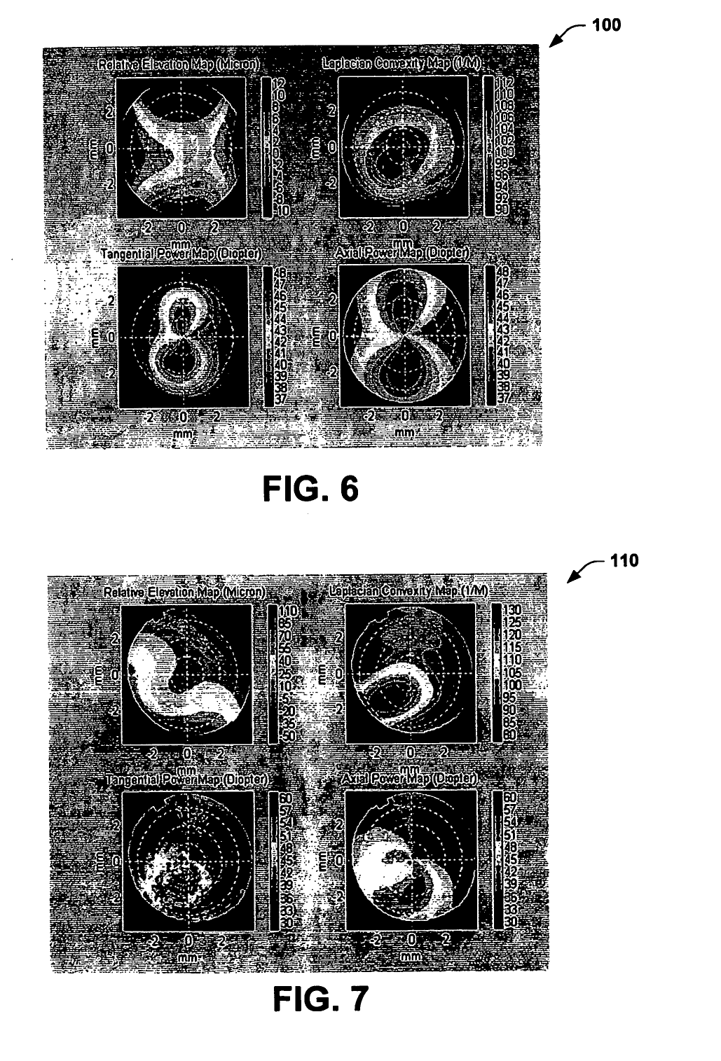

The cone appears broader, distorted and less steep compared to the central cone in all four maps in the map display set, but the convexity map is least affected. FIG. 6 illustrates a map display set 100 of a simulation of an inferior-temporal cone (5D) on 4D with-the-rule astigmatism. FIG. 6 illustrates a map display set 100 that illustrates the introduction of astigmatism to the cornea causing the cones to become partially obscured on all maps of the map display set 100 except the convexity map. FIG. 7 illustrates a map display set 110 of an actual topography from a keratoconic eye. In the corneal topography of the keratoconic eye, the elevation and axial power maps are dominated by off-axis tilt and the cone is poorly defined. Only the convexity map illustrates a clearly defined cone in all four examples.

All conventional corneal topography displays (e.g., relative elevation, axial power, and tangential power) are defined relative to a sphere centered on a central axis. Therefore, the appearance of a cone would change depending on location and axis tilt. The convexity map avoids these problems. The convexity map is also insensitive to regular astigmatism, a non-pathologic condition. Thus, the convexity map facilitates the recognition of shape pathologies such as keratoconus.

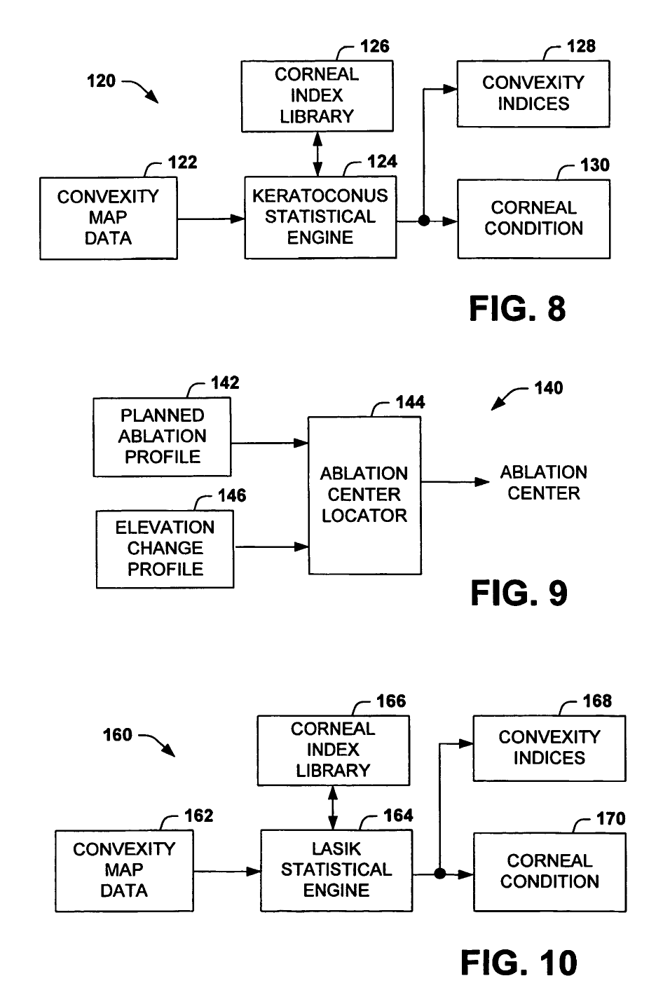

FIG. 8 illustrates a system 120 for determining the presence of keratoconus of a cornea in accordance with an aspect of the present invention. A keratoconus statisitical engine 124 determines one or more statistical parameters associated with a convexity map data set 122. The convexity map data set 122 can be derived by employing corneal elevation map data derived from videokeratography. The keratoconus statistical engine 124 then determines a set of quantative convexity indices 128 employing the one or more statistical parameters. The quantitative set of convexity indices 128 can be employed in detecting keratoconus. The keratoconus statistical engine 124 can determine statistical indices such as the maximum (Max), minimum (Min), median, and inferior and superior hemi-averages (Inf and Sup) that can be compared with the index ranges residing in a corneal index library 126 to determine the presence or absence of keratoconus.

The kerataconus statistical engine 124 can also employ four derivative indices:

Max, Max-Median Difference, Max-Min difference, and Inf-Sup difference that can be compared with the index ranges in the corneal statistic library 126 to determine the presence or absence of keratoconus. Additionally, the keratoconus statistical engine 124 can compare the derived indices with index ranges in a corneal index library 126 to provide a corneal condition 130. The corneal index library 126 includes index ranges associated with various stages of kerataconus in addition to index ranges associated with a cornea without keratoconus. The index ranges can also be provided with the convexity indices 128. A severity level of the keratoconus (e.g., keratoconus, forme fruste keratoconus) can be provided by the kerataconus statistical engine 124.

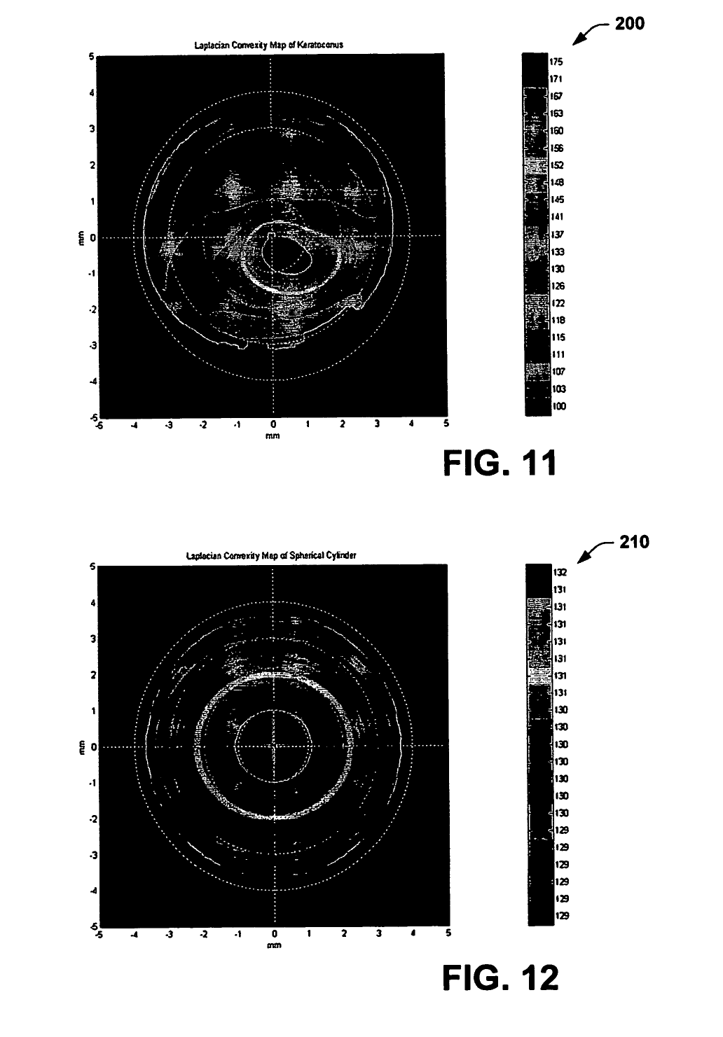

Diagnostics can also be performed by simply reviewing graphical displays of the convexity map to determine the presence or absence of keratoconus. FIGS. 11-12 illustrate graphical displays of a convexity map with and without keratoconus. FIG. 11 illustrates a graphical display 200 of a convexity map of a cornea with keratoconus, while FIG. 12 illustrates a graphical display 210 of a convexity map of a simulated cylinder without keratoconus. Due to the characteristics of the convexity map, the difference between the variation aroused by astigmatism and keratoconus is substantially amplified.

FIG. 9 illustrates a system 140 for locating a center of laser ablation in accordance with an aspect of the present invention. Laser ablation centration is a very important standard in checking the performance of laser eye trackers and alignment devices and its proper usage. Laser ablation for myopia correction causes the central cornea to be flattened out, which can be detected as a central circle lower convexity border by a ring of high convexity. The area and center of the ablation can be easily determined from the ring of high convexity. In hyperopia correction, the ablation is clearly defined by a ring of low convexity. While the ablation center and borders can also be traced from axial or tangential maps, those maps can be substantially affected by a slight tilt of the eye's axis or position relative to the topography system. The convexity map is unaffected by the tilt and therefore more reliable.

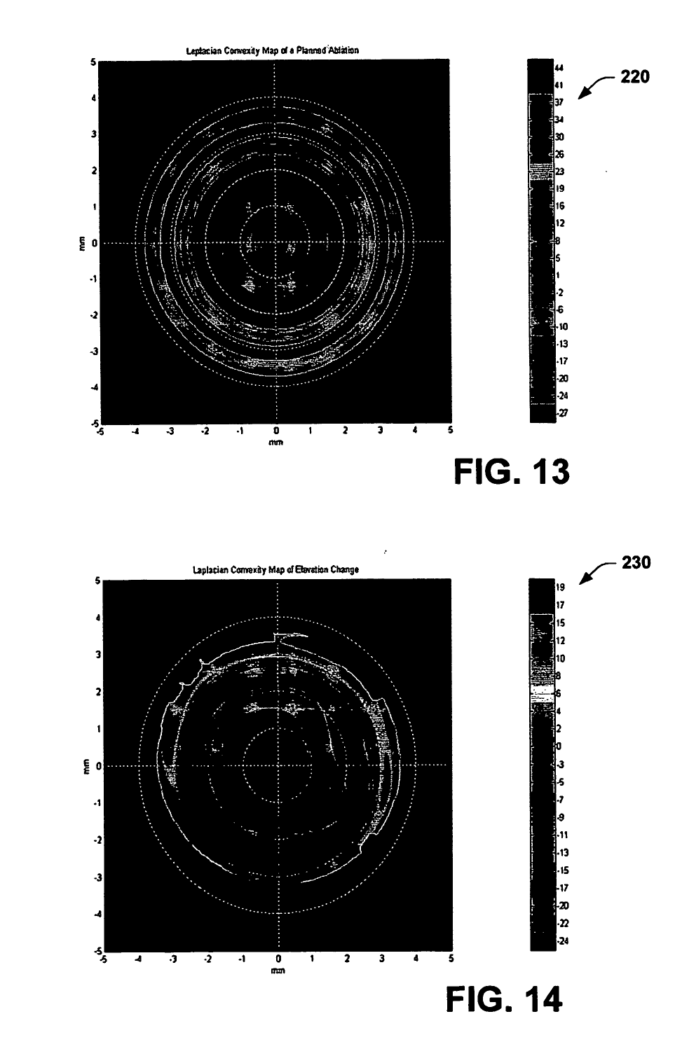

The system 140 includes an ablation center locator 144 that retrieves a planned ablation profile 142 and an elevation change profile 146 and then determines an ablation center. FIG. 13 illustrates a graphical display 220 of a convexity map of a planned ablation depth profile, while FIG. 14 illustrates a graphical display 230 of a convexity map derived from the elevation change map after the ablation. A dramatic change in convexity along the border of the ablation zone can be detected on the convexity map as a clear annular pattern. The ablation center can be determined by searching for maximum cross-correlation between the convexity map of planned ablation profile 142 and the convexity map of the elevation change map 146. Alternatively, the post operative convexity map can be employed instead of the convexity change map because of the similarity of the two maps.

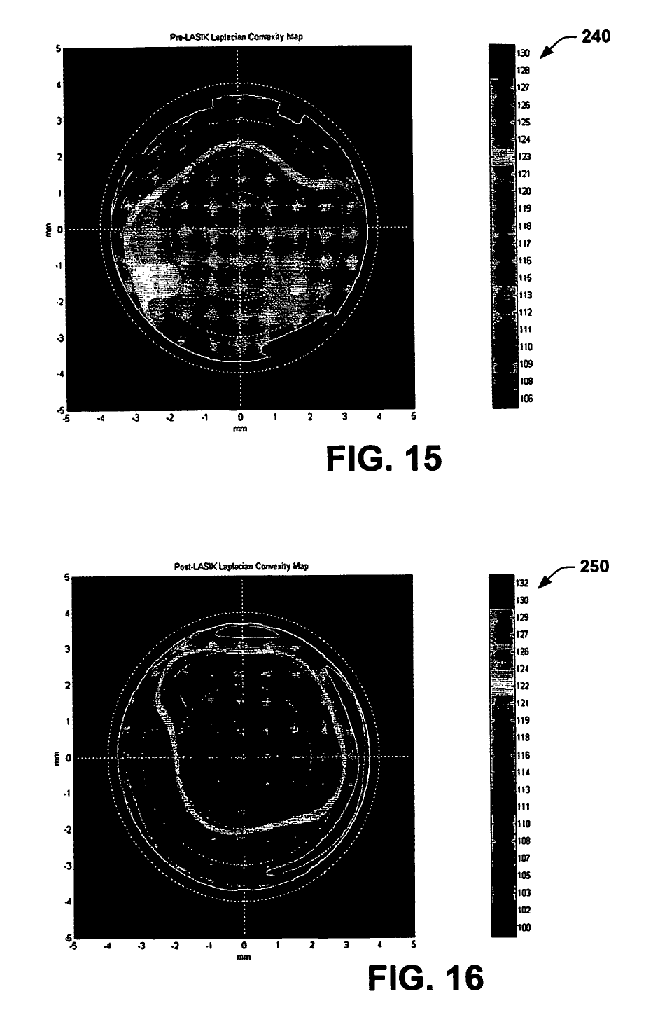

Detection of the presence of previous keratorefractive surgery is useful to the military and other agencies who want to screen out people who had previous eye surgery from certain special jobs (such as pilots). It is conventionally determined by visual inspection of axial or tangential maps. The convexity map can be employed to detect laser corneal ablation. Laser corneal ablation will cause considerable variance in convexity. Due to its special feature of emphasis on the elevation gradient, its variance will be greater than the variance of axial or tangential power maps. FIG. 15 illustrates a graphical display 240 of a convexity map of a normal Pre-Lasik patient, and FIG. 16 illustrates a graphical display 250 of a convexity map of a normal Post-LASIK patient in which the flattening in the central region is readily detectable by convexity map. Quantitative parameters can be derived from convexity map of corneal surface to realize automatic detection of presence of laser corneal ablation. For example, following the same classification approach of keratoconus and normal group, parameters can be derived to differentiate post-op from pre-op LASIK employing the convexity map. Statistical parameters including the maximum (Max), minimum (Min), median, and inferior and superior hemi-averages (Inf and Sup) can be employed to detecting previous keratorefractive surgery.

LASIK and other keratorefractive surgeries in general modify the central curvature of the cornea more than the curvature of the peripheral cornea. After correction for hyperopia (farsightedness), the central cornea becomes more convex. After correction for myopia (nearsightedness), the central cornea becomes less convex. By taking the difference between the convexity of the central cornea and the peripheral cornea, keratorefractive surgery can be detected by noting abnormaly high or low values. Besides the center-periphery difference, location indepent parameters such as the Max-Median, Max-Min differences in the convexity map can be employed to detect keratorefractive surgery. Statistical threshold can be derived for these indices to be employed in automatic detection of presence of laser and other modes of keratorefractive surgery from the corneal topography of an eye.

FIG. 10 illustrates a system 160 for detecting the presence of previous keratorefractive surgery of a cornea in accordance with an aspect of the present invention. A LASIK statisitical engine 164 determines one or more statistical parameters associated with a convexity map data set 162. The LASIK statistical engine 164 determines a set of quantative indices 168 employing the one or more statistical parameters. The quantitative set of convexity indices 168 can be employed in detecting previous keratorefractive surgery. The LASIK statistical engine 168 can determine statistical indices such as the maximum-Medium, Max-Min differences, central-peripheral corneal difference in the Laplacian map. The LASIK statistical engine 168 can then compare the derived indices with index ranges residing in a corneal index library 166 to determine the presence or absence of previous keratorefractive surgery. Additionally, the LASIK statistical engine 168 can compare the derived indices with index ranges in the corneal index library 166 to provide a corneal condition 170. The index ranges can also be provided with the convexity indices 168.

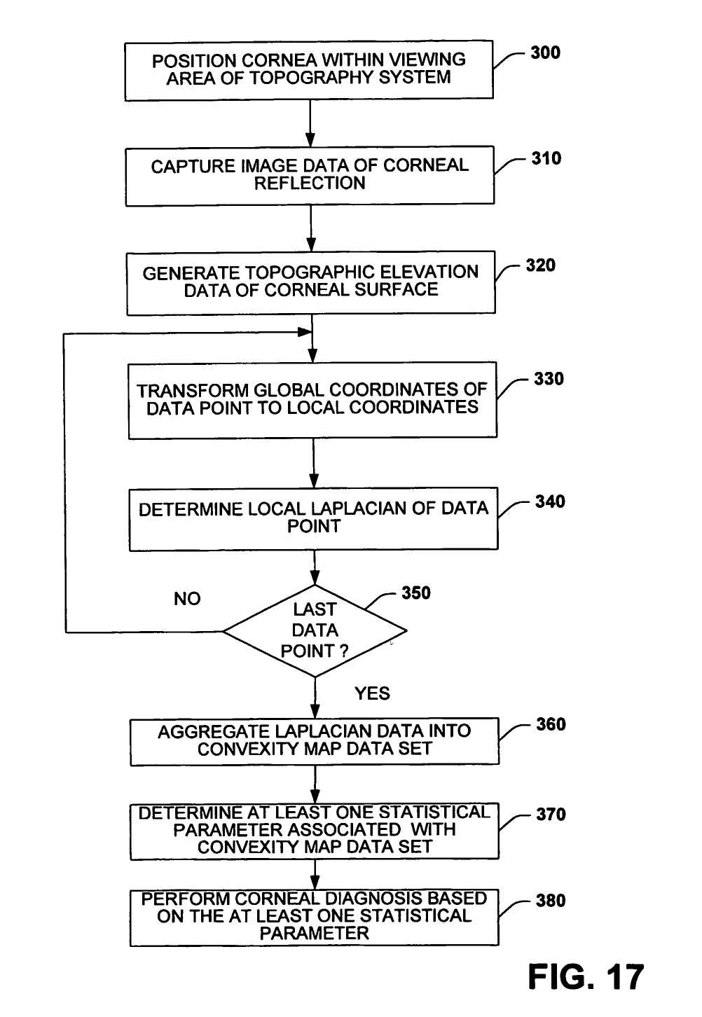

In view of the foregoing structural and functional features described above, a methodology in accordance with various aspects of the present invention will be better appreciated with reference to FIG. 17. While, for purposes of simplicity of explanation, the methodology of FIG. 17 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect the present invention.

FIG. 17 illustrates a methodology for diagnosing a condition associated with a cornea in accordance with an aspect of the present invention. The methodology begins at 300 where a cornea of a patient is positioned within a viewing area of a topography system. At 310, image data of the cornea is captured, for example, by reflecting light off the cornea through a mask (e.g., placido ring), and capturing the reflected image data by an electronic camera (e.g., CCD camera). At 320, topographic elevation data of the cornea is generated employing the reflected image data. The topographic elevation data can be generated directly by the reflected image data, or be converted from axial radius data that is received from the camera. The methodology then proceeds to 330. At 330, the global coordinates of a data point from the elevation data is converted to local coordinates. The global coordinates of the data point can be converted to the local coordinates by employing the transform matrix of M illustrated in EQ. 5. The methodology then proceeds to 340. At 340, the Laplacian of the local coordinates of the data point is computed to provide the convexity at that data point. The local data point can be convolved with a kernel, such as the one illustrated in EQ. 6. The methodology then proceeds to 350.

At 350, the methodology determines if the convexity of the last data point has been determined. If the convexity of the last data point has not been determined (NO), the methodology returns to 330 to transform the next data point from global to local coordinates, and to determine the convexity of the local coordinates. If the convexity of the last data point has been determined (YES), the methodology proceeds to 360. At 360, the convexity data is aggregated into a single convexity map data set. At 370, at least one statistical parameter associated with the convexity map data set is determined. A corneal diagnosis is then performed based on the at least one statistical parameter at 380.

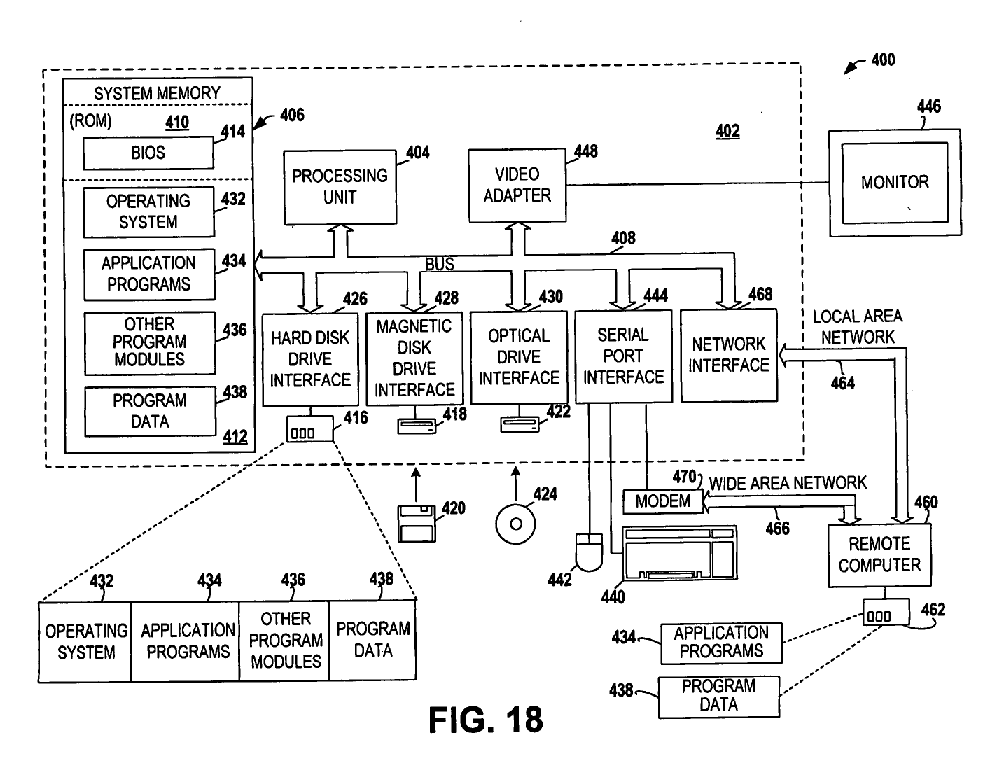

In order to provide additional context for implementing various aspects of the present invention, FIG. 18 and the following discussion are intended to provide a brief, general description of a suitable computing environment 400 in which the various aspects of the present invention may be implemented. While the invention has been described above in the general context, the invention can employ computer-executable instructions of a computer program that runs on a local computer and/or remote computer. Those skilled in the art will recognize that the invention also may be implemented in combination with other program modules. Moreover, those skilled in the art will appreciate that the inventive methods may be practiced with other computer system configurations, including single-processor or multiprocessor computer systems, minicomputers, mainframe computers, as well as personal computers, hand-held computing devices, microprocessor-based or programmable consumer electronics, and the like, each of which may operatively communicate with one or more associated devices. The illustrated aspects of the invention may also be practiced in distributed computing environments where certain tasks are performed by remote processing devices that are linked through a communications network. However, some, if not all, aspects of the invention may be practiced on stand-alone computers.

With reference to FIG. 18, an exemplary system environment 400 for implementing the various aspects of the invention includes a conventional computer 402, including a processing unit 404, a system memory 406, and a system bus 408 that couples various system modules, including the system memory, to the processing unit 404. The processing unit 404 may be any commercially available or proprietary processor. In addition, the processing unit may be implemented as multi-processor formed of more than one processor, such as may be connected in parallel.

The system bus 408 may be any of several types of bus structure including a memory bus or memory controller, a peripheral bus, and a local bus using any of a variety of conventional bus architectures such as PCI, VESA, Microchannel, ISA, and EISA, to name a few. The system memory 406 includes read only memory (ROM) 410 and random access memory (RAM) 412. A basic input/output system (BIOS) 414, containing the basic routines that help to transfer information between elements within the computer 402, such as during start-up, is stored in ROM 410.

The computer 402 also may include, for example, a hard disk drive 416, a magnetic disk drive 418, e.g., to read from or write to a removable disk 420, and an optical disk drive 422, e.g., for reading from or writing to a CD-ROM disk 424 or other optical media. The hard disk drive 416, magnetic disk drive 418, and optical disk drive 422 are connected to the system bus 408 by a hard disk drive interface 426, a magnetic disk drive interface 428, and an optical drive interface 430, respectively. The drives 416-422 and their associated computer-readable media provide nonvolatile storage of data, data structures, computer-executable instructions, etc. for the computer 402. Although the description of computer-readable media above refers to a hard disk, a removable magnetic disk and a CD, it should be appreciated by those skilled in the art that other types of media which are readable by a computer, such as magnetic cassettes, flash memory cards, digital video disks, Bernoulli cartridges, and the like, can also be used in the exemplary operating environment 400, and further that any such media may contain computer-executable instructions for performing the methods of the present invention.

A number of program modules may be stored in the drives 416-422 and RAM 412, including an operating system 432, one or more application programs 434, other program modules 436, and program data 438. The operating system 432 may be any suitable operating system or combination of operating systems. By way of example, the application programs 434 and program modules 436 can routines for transforming corneal image data to elevation data and transforming the elevation data into corneal convexity data. Additionally, the application programs 434 and program modules 436 can include routines for providing selection of different corneal topographic map displays (e.g., axial, tangential, elevation, convexity) and indices for diagnosis of defects (e.g., keratoconus, keratorefractive surgery) in addition to other corneal information (e.g., laser ablation center). One or more statistical parameters associated with the convexity map data set can be employed to provide automatic diagnosis of the cornea.

A user can enter commands (e.g., capture, display) and information (e.g., selection type) into the computer 402 through one or more user input devices, such as a keyboard 440 and a pointing device (e.g., a mouse 442). Other input devices (not shown) may include a microphone, a joystick, a game pad, a satellite dish, wireless remote, a scanner, or the like. These and other input devices are often connected to the processing unit 404 through a serial port interface 444 that is coupled to the system bus 408, but may be connected by other interfaces, such as a parallel port, a game port or a universal serial bus (USB). A monitor 446 or other type of display device is also connected to the system bus 408 via an interface, such as a video adapter 448. In addition to the monitor 446, the computer 402 may include other peripheral output devices (not shown), such as speakers, printers, etc.

It is to be appreciated that the computer 402 can operate in a networked environment using logical connections to one or more remote computers 460. The remote computer 460 may be a workstation, a server computer, a router, a peer device or other common network node, and typically includes many or all of the elements described relative to the computer 402, although, for purposes of brevity, only a memory storage device 462 is illustrated in FIG. 18. The logical connections depicted in FIG. 18 may include a local area network (LAN) 464 and a wide area network (WAN) 466. Such networking environments are commonplace in offices, enterprise-wide computer networks, intranets and the Internet.

When used in a LAN networking environment, for example, the computer 402 is connected to the local network 464 through a network interface or adapter 468. When used in a WAN networking environment, the computer 402 typically includes a modem (e.g., telephone, DSL, cable, etc.) 470, or is connected to a communications server on the LAN, or has other means for establishing communications over the WAN 466, such as the Internet. The modem 470, which can be internal or external relative to the computer 402, is connected to the system bus 408 via the serial port interface 444. In a networked environment, program modules (including application programs 434) and/or program data 438 can be stored in the remote memory storage device 462. It will be appreciated that the network connections shown are exemplary and other means (e.g., wired or wireless) of establishing a communications link between the computers 402 and 460 can be used when carrying out an aspect of the present invention.

It will be appreciated that operation of the invention can include the manipulation by the processing unit 404 of electrical signals representing data bits which causes a resulting transformation or reduction of the electrical signal representation, and the maintenance of data bits at memory locations in the memory system (including the system memory 406, hard drive 416, floppy disks 420, CD-ROM 424, and remote memory 462 ) to thereby reconfigure or otherwise alter the computer system's operation, as well as other processing of signals. The memory locations where such data bits are maintained are physical locations that have particular electrical, magnetic, or optical properties corresponding to the data bits.

What has been described above are examples of the present invention. It is, of course, not possible to describe every conceivable combination of modules or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

Having described the invention, the following is claimed:

1. A corneal topography system comprising:
an optical assembly that captures an image of a cornea and provides corneal image data; and
a diagnostic system that converts the image data to elevation map data, the diagnostic system having a convexity module that transforms the elevation map data to convexity map data.

2. The system of claim 1, the convexity module transforms the elevation map data from global data to local data, the local data is then convolved to provide convexity map data associated with the image data of the cornea.

3. The system of claim 2, the convexity module transforms the elevation data from global data to local data by employing a transform matrix M comprising:
$$M = [ \begin{matrix}\frac{{N}_{x} {N}_{z}}{\sqrt{{N}_{x}^{2} + {N}_{y}^{2}}} & \frac{{N}_{y} {N}_{z}}{\sqrt{{N}_{x}^{2} + {N}_{y}^{2}}} & - \sqrt{{N}_{x}^{2} + {N}_{y}^{2}} \\ - \frac{{N}_{y}}{\sqrt{{N}_{x}^{2} + {N}_{y}^{2}}} & \frac{{N}_{x}}{\sqrt{{N}_{x}^{2} + {N}_{y}^{2}}} & 0 \\ {N}_{x} & {N}_{y} & {N}_{z}\end{matrix} ]$$
where (Nₓ, N_{Y}, N_{z}) denotes the local surface normal.

4. The system of claim 2, the convexity module transforms the local elevation data to convexity data by convoluting the local data points with a kernel matrix comprising:
$${f}_{k} = \frac{1}{( 6 * {d}^{2} )} [ \begin{matrix}0.5 & 2 & 0.5 \\ 2 & - 10 & 2 \\ 0.5 & 2 & 0.5\end{matrix} ]$$
where d is the distance between two adjacent points on a one of a digital mesh and digital matrix that represent the map data set.

5. The system of claim 1, the diagnostic system further comprising a diagnostic assessment system that determines a condition associated with the cornea based on the convexity map data.

6. The system of claim 5, the condition being keratoconus and the diagnostic system providing a severity level of the keratoconus.

7. The system of claim 5, the diagnostic system determines at least one statistical parameter associated with the convexity map to derive convexity indices associated with the convexity map data, the diagnostic system employs the convexity indices to determine a condition associated with the cornea.

8. The system of claim 5, the condition being previous kerorefractive surgery.

9. The system of claim 1, the diagnostic system operative to determine an ablation center of laser ablation of the cornea by determining the maximum cross-correlation between a convexity map of a planned ablation profile and one of a convexity map of an elevation change profile and a post operative convexity map.

10. The system of claim 1, further comprising a display, the diagnostic system operative to graphical display the convexity map data set on the display.

11. A system for diagnosing a condition of a cornea, the system comprising:
a coordinate transformation module that transforms elevation data points associated with surface measurements of a cornea from global coordinates to local coordinates;
a convolution module that transforms the local coordinates of the elevation data point to local convexity map data; and
a diagnostic system that employs the local convexity data in diagnosis of a condition of the cornea.

12. The system of claim 11, further comprising a display, the diagnostic system operative to graphical display the convexity map data set on the display.

13. The system of claim 11, the diagnostic system further comprising a statistical engine that determines at least one parameter of the convexity map data and provides indices associated with the at least one parameter, the indices being employed to determine the condition of the cornea.

14. The system of claim 13, the indices being at least one of maximum, minimum, median, inferior hemi-averages, superior hemi-averages, max-median-difference, max-min difference and inferior-superior difference.

15. The system of claim 11, the condition of the cornea being one of a normal cornea, a cornea with keratoconus and a cornea with previous kerorefractive surgery.

16. The system of claim 11, the convexity module performing a convexity of the elevation data points, the convexity being represented as:
$$- {∇}^{2} h = - ( \frac{{\partial }^{2} h}{\partial {x}^{2}} + \frac{{\partial }^{2} h}{\partial {y}^{2}} )$$
where ∇² is the Laplacian operation in two dimension x and y, and where h represents the corneal surface elevation along the z axis and (x, y, z) is the local rectangular coordinate system with origin at a point on the corneal surface with z being the normal to the corneal surface.

17. A method for providing corneal surface measurements, the method comprising:
receiving image data of a cornea;
generating an elevation map data set of the cornea corresponding to the received image data; and
transforming the elevation map data set to a convexity map data set.

18. The method of claim 17, the transforming the elevation map data set to a convexity map data set comprising performing a Laplacian operation on elevation data points to provide a convexity value corresponding to each elevation data point, the convexity being computed as the negative of the Laplacian of the local elevation.

19. The method of claim 17, further comprising determining at least one statistical parameter associated with the convexity map data set, deriving indices from the at least one statistical parameter and employing the derived indices in diagnosis of a condition of the cornea.

20. The method of claim 19, the indices being at least one of maximum, minimum, median, inferior hemi-averages, superior hemi-averages, max-median difference, max-min difference and inferior-superior difference.

21. The method of claim 17, further comprising diagnosing a corneal condition employing the convexity map data set.

22. The method of claim 21, the condition of the cornea being one of a normal cornea, a cornea with keratoconus and a cornea with previous kerorefractive surgery.

23. A computer readable medium having computer-executable instructions for performing the method of claim 17.

24. A system for diagnosing a condition of a cornea, the system comprising:
means for capturing image data of a cornea;
means for generating elevation map data corresponding to the captured image data;
means for transforming the elevation map data set to convexity map data;
means for deriving indices employing at least one statistical parameter of the convexity map data; and
means for diagnosing a condition of the cornea based on the indices.

25. The system of claim 24, the means for transforming the elevation map data set to convexity map data comprising means for transforming the elevation map data from global data to local data, and means for convolving the local data to provide convexity map data associated with the image data of the cornea.
